(19) United States
(12) Patent Application Publication
(10) Pub. No.: US 2020/0347335 A1
(43) Pub. Date: Nov. 5, 2020

(54) APPARATUS ASSEMBLY FOR CARRYING OUT AT LEAST ONE BASIC OPERATION IN A BIOPHARMACEUTICAL PROCESS

(71) Applicant: Sartorius Stedim Biotech GmbH, Goettingen (DE)

(72) Inventor: Stefan SCHLACK, Goettingen (DE)

(21) Appl. No.: 16/770,098

(22) PCT Filed: Nov. 30, 2018
(86) PCT No.: PCT/EP2018/083101

§ 371 Date: Jun. 5, 2020

(30) Foreign Application Priority Data
Dec. 15, 2017 (DE) 10 2017 130 093.7

(51) Int. Cl. C12M 3/00; C12M 1/00; C12M 1/34

(52) U.S. Cl. CPC C12M 23/48; C12M 41/00; C12M 23/52; C12M 23/46

(57) ABSTRACT

A device arrangement for performing at least one unit operation in a biopharmaceutical process comprises a central operating module having a control means. The device arrangement further comprises a process plate which is provided for attachment to the operating module. On the process plate, process components which are essential for carrying out the unit operation can be pre-assembled.

The invention relates to a device arrangement for performing at least one unit operation in a biopharmaceutical process.

In general, processes in which single-use devices are used are becoming increasingly widespread in the pharmaceutical production of high-quality active substances due to the high flexibility that can be achieved and the savings in time, investment and operating expenses such as cleaning and inspection. It can be assumed that in the future, several biopharmaceutical products will be produced in a single plant. Furthermore, it is becoming increasingly important for the manufacturers of biopharmaceutical products to be able to quickly adapt capacities to market demand. This means that process volumes should be flexibly adaptable.

In biopharmaceutical processes, prefabricated standardized device arrangements which are adapted to be configured in many ways are often used to perform specific unit operations. One example of such a solution is the FlexAct® System from Sartorius Stedim Biotech GmbH, which is flexibly adaptable and can be used, among other things, for buffer preparation, cell collection, virus inactivation, media preparation, virus depletion, line testing or bag testing. The core of this device arrangement is a compact central multifunctional operating module in the form of a stainless steel trolley having a control unit and an operating panel. Using the FlexAct® System, specific unit operations using selected single-use process components (bags, hose lines, connectors, filter capsules, etc.) can be performed in an at least partially automated manner.

The device arrangements known from the prior art are subject to certain restrictions. Usually, a device arrangement is designed for a specific process step with a specific volume. Installation or conversion of the device arrangement on the part of the user can only be carried out with great effort, which leads to an increased susceptibility to errors. In this context, in particular the fast and correct connection of single-use process components is a challenge. Furthermore, it is necessary to perform an adjustment between the basic components of the device arrangement (e.g. a pump) with the single-use process components connected thereto and further used single-use process components (single-use assemblies, “wetware”), and the control software (selected process step, basic conditions).

The object of the invention is to overcome the limitations of the device arrangements known from the prior art.

This object is achieved by a device arrangement for performing at least one unit operation in a biopharmaceutical process, having the features of claim 1. Advantageous and useful configurations of the device arrangement according to the invention are indicated in the sub-claims.

The device arrangement according to the invention for performing at least one unit operation in a biopharmaceutical process comprises a central operating module which has a control means. The device arrangement further comprises a process plate which is provided for attachment to the operating module. Process components which are essential for performing the unit operation can be pre-assembled on the process plate.

The invention is based on the findings that the process components essential for performing a unit operation in a biopharmaceutical process can be mounted very clearly on a suitable plate. The provision of an independent process plate according to the invention makes it possible to create multifunctional device arrangements in a simple manner, which are both easy to install and allow rapid adaptation to different process steps. Since the process components can be pre-assembled by the manufacturer in a desired configuration, the corresponding effort on the part of the user is eliminated. In addition, errors in the assembly and mounting of the process components on the part of the user side are eliminated.

The invention is particularly suitable and provided for process steps in which single-use components are used. Accordingly, the process components which can be pre-assembled on the process plate are preferably single-use components.

In particular hose lines, filters, valves or sensors are regarded as “essential” process components for performing a unit operation in a biopharmaceutical process which can be pre-assembled on the process plate.

In order to keep the mounting of the process plate itself as simple as possible, one or more plug connections are provided in a preferred embodiment of the invention, by which the process plate can be mounted on the operating module.

Plug connections that define a specific position and orientation of the process plate are particularly preferred. This clearly determines the position and orientation of all process components mounted on the process plate, which considerably simplifies the connection or the linking of the process components to the basic components of the device arrangement (e.g. a pump).

Preferably, holders, such as hose clamps, valve and sensor holders, which are adapted to be detachably mounted at various locations on the process plate are provided for fastening the process components on the process plate. Due to the flexible arrangement of the holders, the process components can be optimally positioned depending on the configuration.

As an aid to the user, colored markings can be provided on the process plate, which clearly show the user the course of liquid flows, for example.

According to a particular embodiment of the invention, the process plate is foldable. This enables a space-saving transport or space-saving storage of the process plate. A design in which the process plate protects the process components mounted thereon when it is in the folded state is also advantageous.

The process plate provided according to the invention is preferably provided with an identification that is linked to a predefined configuration of the process plate. This means that after reading the identification, the control means of the device arrangement knows the exact configuration of the process plate (which process components are arranged and connected and how they are arranged and connected). The identification may be a barcode, a two-dimensional code (e.g. a QR code), an RFID tag or the like.

According to a further development of this aspect, the control means is set up such that it automatically performs a comparison between at least one basic component of the device arrangement and a process component mounted on the process plate and a control software of the device arrangement on the basis of the identification. Here, comparison means that it is in particular checked whether the basic component of the device arrangement is compatible with the process component on the process plate. If this is the case, the operating parameters of the basic component, if necessary, are automatically adapted to the process component on the process plate or generally to the basic conditions of the unit operation to be performed, which are determined by the recognized process plate.

For a very specific spatial arrangement of a basic component of the device arrangement (e.g. a pump), which may be required for connection or linking to a process component mounted on the process plate, the invention provides at least one holding device for holding a basic component, which is adapted to be adjusted such that the basic component can be brought into a predefinable position and orientation, which is provided for connection or linking of the basic component to a process component mounted on the process plate.

An electromechanical or electrohydraulic adjusting means is preferably provided for adjusting the holding device. Such adjusting means may be electronically driven and regulated by the control means of the device arrangement so that they bring the holding device precisely into the desired position.

Preferably, the control means is set up such that it drives the adjusting device on the basis of the previously explained identification of the process plate such that the holding device is automatically brought into the intended position and orientation. This considerably reduces the effort for commissioning on the part of the user.

A holding device provided to hold a basic component of the device arrangement may also be designed as a replaceable holding device. The control means is then preferably adapted such that it recognizes on the basis of the identification of the process plate whether it is necessary to replace a basic component of the device arrangement. If this is the case, it then issues a corresponding note for the user.

With this measure, faulty installations with incompatible components may be avoided.

A further development of the invention provides a table or carriage separate from the central operating module, which has a rest supported by rollers. Such a rest serves to assist the mounting and/or demounting of a holding device (e.g. a board) located on the rest along with a basic component held thereby to or from a frame of the device arrangement. Due to the rollers, the holding device along with the basic component can be easily pushed to a designated position for clipping the holding device on the frame.

With a height-adjustable rest of the table or carriage, it is possible to achieve clip-on positions of the device arrangement at different heights on the frame.

A further measure to facilitate the mounting of a basic component to the device arrangement is to equip a holding device designed as a board with rollers to hold a basic component. The basic component to be mounted can then be easily pushed into the correct connection position after it has been placed on the board.

In addition, the holding device equipped with rollers may also be provided with guide rails for the basic component and further optionally with a slider by which the basic component can be pushed automatically against the guide rail.

Further features and advantages of the invention will become apparent from the description below and from the attached drawings to which reference is made. In the drawings:

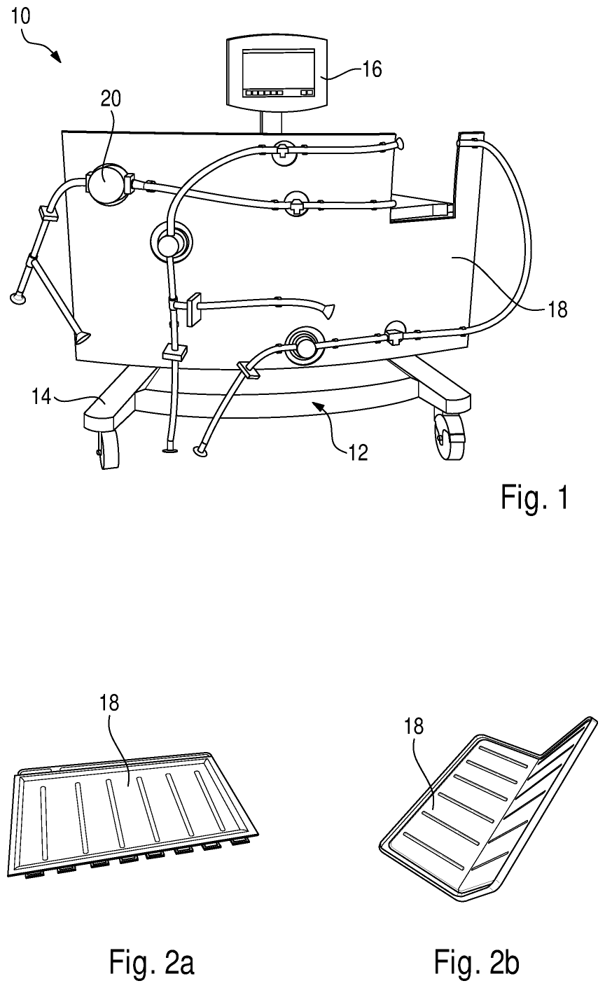

FIG. 1 shows a device arrangement according to the invention for performing a unit operation in a biopharmaceutical process;

FIGS. 2a and 2b show an unloaded process plate for a device arrangement according to the invention in the unfolded and folded state, respectively;

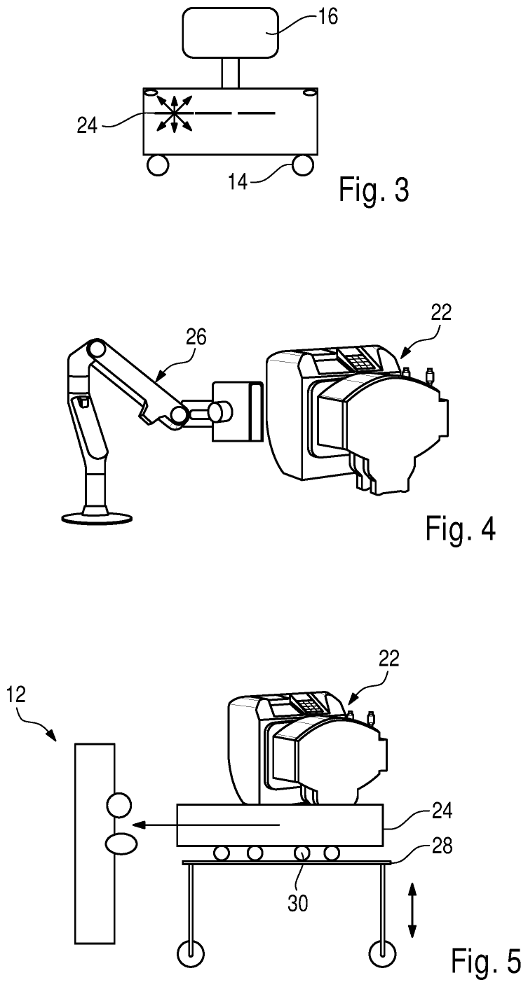

FIG. 3 schematically shows a device arrangement according to the invention (without process plate) having a movable holding device;

FIG. 4 schematically shows a movable holding device in the form of a hydraulic arm for a device arrangement according to the invention; and

FIG. 5 schematically shows a roller carriage as an aid for mounting a basic component on a device arrangement according to the invention.

FIG. 1 shows a device arrangement 10 adapted to be configured in various ways for performing specific unit operations in biopharmaceutical processes. The device arrangement 10 comprises a compact central multifunctional operating module 12 in the form of a stainless steel trolley 14 having a control means (not shown) and an operating panel 16. A special component of the device arrangement 10 is a separate, loadable process plate 18, which will be described in more detail below.

The process plate 18 is a flat, largely flat structure that can be easily mounted to a frame of the device arrangement 10, e.g. by one or more plug connections. The plug connection components on the frame and on the process plate 18 are arranged and adapted to each other such that the process plate 18 is mounted in a predefined position and orientation.

On request, the process plate 18 is provided as a separate unit in a desired configuration, in which all process components necessary for a desired process step are pre-assembled and, if necessary, connected to each other (assembly). The process components may be hose lines, filters, valves, sensors etc., which are designed for use as single-use components.

Holders 20 (hose clamps, valve and sensor holders, etc.) are provided for fastening the process components to the process plate 18 and can in turn themselves be flexibly and detachably mounted to the process plate 18 using plug connectors. For this purpose, punched holes or similar are provided in the process plate 18 in which the connectors can engage.

In this way, a suitable component arrangement for a desired process step can be quickly preconfigured on the process plate 18. Due to the flexibly positionable and detachably mountable holders 20, a change of the configuration or the fitting for performing another process step is possible without much effort.

The holders 20 for the process components of a specific configuration are in particular arranged on the process plate 18 such that optimum guidance of the hose lines is ensured. Due to the optimized guidance of the hose lines, the standing volume can be reduced in order to ensure that the hose lines run empty with as few residues as possible. For an effective self-draining after the process step has been carried out, the hose lines, if required, can also be moved and fixed in a draining position without much effort.

In addition, the liquid flows predefined by the configuration can be indicated in color on the process plate 18.

As shown symbolically in FIGS. 2a and 2b, the process plate 18 is foldable to simplify the handling and logistics thereof. The process plate 18 can be configured such that the process plate 18, when folded, also provides transport protection for already mounted process components.

In addition, the process plate 18 can be provided with an identification in the form of a barcode, a two-dimensional code (e.g. QR code), an RFID tag or similar. The identification is linked to the predefined configuration of the process plate 18. By entering the identification (automatic reading or scanning or manual entry), an automatic comparison of basic components of the device arrangement 10 with the process components installed on the process plate 18 and the control software of the operating module of the device arrangement 10 can thus be carried out in a simple manner. The control software can access data provided by an MES or SCADA system or similar.

The process plate 18 can be so large and equipped with so many holders 20 that several assemblies can be mounted on the same process plate 18. In general, the process plate 18 can be pre-installed by the manufacturer or installed and, if necessary, converted by the user, depending on the user's wishes.

Depending on the configuration of the process plate 18, it may be necessary to bring specific basic components 22 of the device arrangement 10, which are not mounted on the process plate 18, into the correct position and orientation (pose), so that a correct and safe linking or a correct and safe interaction of these basic components 22 with the process components on the process plate 18 is ensured. For this purpose, the device arrangement 10, as symbolically shown in FIG. 3, can be provided with movable boards or other holding devices 24 by which pumps, motors, etc., for example, can be brought into the correct pose. An adjusting means is provided for the mobility of a holding device 24, which enables movements in the required degrees of freedom (linear and/or rotary movements, possibly in different directions).

The alignment of a holding device 24 can be performed manually or in an automated manner by an electromechanical or hydraulic adjusting means, such as a hydraulic arm 26 shown symbolically in FIG. 4. In particular, after (automated) input of the identification of the process plate 18, the control means of the device arrangement 10 can automatically bring the holding device(s) 24 into the correct pose by the adjusting device, accessing corresponding data from an MES or SCADA system or similar.

Furthermore, it is possible to replace individual holding devices 24, e.g. a board, in order to install a pump of a different size, for example. If after identification of the process plate 18 within the scope of a previously described comparison, the control means recognizes that such a replacement is necessary, a corresponding note can be issued to the operating personnel.

A table or carriage 28 can be used as an aid for installing or replacing a heavy or bulky basic component 22 on the device arrangement 10, as symbolically shown in FIG. 5. On the table or carriage 28, there are rollers 30 on which the holding device 24 (e.g. a clip-on panel) to be mounted rests along with the basic component 22 attached thereto (e.g. a heavy pump). The table or carriage 28 is then pushed close to the clip-on position for the holding device 24, and the holding device 24 with the basic component 22 is again pushed to the clip-on position using the rollers 30, such that a simple mounting is possible. The rest of the table or carriage 28 is preferably adjustable in height in order to make clip-on positions at different heights accessible. The table or carriage 28 can also be used to demount a holding device 24 with the basic component 22 attached thereto.

A further possibility to simplify the mounting of a basic component to the device arrangement 10 is the provision of rollers which are, if necessary retractable and extendable, on a board 24 (fixedly or detachably attached to the frame of the device arrangement 10). The basic component 22 to be mounted is then first placed on the board 24 and can then be brought into the correct connection position. For a further simplification, guide rails can also be extended to bring the basic component 22 to be mounted into the correct position. In addition, a slider can be provided which automatically pushes the basic component 22 against the guide rail.

### LIST OF REFERENCE NUMERALS

10 device arrangement
12 operating module
14 trolley
16 operating panel
18 process plate
20 holders
22 basic component
24 holding device
26 hydraulic arm
28 table or carriage
30 rollers

1. A device arrangement for performing at least one unit operation in a biopharmaceutical process, having a central operating module which includes a control means,
characterized by a process plate which is provided for attachment to the operating module and on which process components which are essential for performing the at least one unit operation can be pre-assembled.

2. The device arrangement according to claim 1, characterized in that the pre-assembled process components are single-use components.

3. The device arrangement according to claim 1, characterized in that the pre-assembled process components include at least one of the following components: hose line, filter, valve, sensor.

4. The device arrangement according to claim 1, characterized in that the process plate is adapted to be mounted to the operating module by one or more plug connections.

5. The device arrangement according to claim 4, characterized in that the one or more plug connections define a specific position and orientation of the process plate.

6. The device arrangement according to claim 1, characterized in that for fastening the process components on the process plate, holders are provided which are adapted to be detachably mounted at different locations on the process plate.

7. The device arrangement according to claim 1, characterized in that colored markings are provided on the process plate.

8. The device arrangement according to claim 1, characterized in that the process plate is foldable.

9. The device arrangement according to claim 1, characterized in that the process plate is provided with an identification which is linked to a specified configuration of the process plate.

10. The device arrangement according to claim 9, characterized in that the control means is set up so as to automatically perform a comparison, on the basis of the identification, between at least one basic component of the device arrangement and a process component mounted on the process plate and a control software of the device arrangement.

11. The device arrangement according to claim 1, characterized by at least one holding device for holding a basic component of the device arrangement the at least one holding device being adjustable such that the basic component can be brought into a position and orientation which is provided for connecting or linking the basic component to a process component mounted on the process plate.

12. The device arrangement according to claim 11, characterized in that an electromechanical or electrohydraulic adjusting means is provided for adjusting the holding device.

13. The device arrangement according to claim 12, characterized in that the control means is set up such that it drives the electrohydraulic adjusting means on the basis of the identification of the process plate such that the at least one holding device is automatically brought into an intended position and orientation.

14. The device arrangement according to claim 1, characterized by a replaceable holding device for holding a basic component of the device arrangement the control means being adapted so as to recognize when it is necessary to replace a basic component of the device arrangement on the basis of the identification of the process plate and then to issue a note.

15. The device arrangement according to claim 1, characterized by a table or carriage separate from the central operating module and having a rest supported by rollers to assist the mounting and/or demounting of a holding device located on the rest along with a basic component held thereby to or from a frame of the device arrangement.

16. The device arrangement according to claim 15, characterized in that the rest is height-adjustable.

17. The device arrangement according to claim 1, characterized in that a holding device configured as a board for holding a basic component of the device arrangement is equipped with rollers.

18. The device arrangement according to claim 17, characterized in that guide rails for the basic component and optionally a slider by which the basic component can be automatically pushed against the guide rail are provided on the holding device (24).
